(19) United States
(12) Patent Application Publication
(10) Pub. No.: US 2015/0150826 A1
(43) Pub. Date: Jun. 4, 2015

(54) ELECTROLYTE IMPREGNATED TIMED-RELEASE STICK-ON PATCH

(71) Applicant: Gregory E. Bator, Jersey City, NJ (US)

(72) Inventor: Gregory E. Bator, Jersey City, NJ (US)

(21) Appl. No.: 14/093,247

(22) Filed: Nov. 29, 2013

(51) Int. Cl. A61K 9/70; A61K 33/14; A61K 31/375; A61K 31/59; A61K 31/185; A61K 31/7016; A61K 33/04; A61K 33/42; A61K 31/4415; A61K 31/7004; A61K 31/194; A61K 31/522; A61K 33/24

(52) U.S. Cl. CPC A61K 9/7084; A61K 31/522; A61K 33/14; A61K 31/375; A61K 31/59; A61K 31/185; A61K 33/24; A61K 33/04; A61K 33/42; A61K 31/4415; A61K 31/7004; A61K 31/194; A61K 31/7016

(57) ABSTRACT

An adhesive patch applied to a person's skin much like a bandage, comprising an adhesive underside. Affixed to the adhesive underside is a reservoir in contact with the skin, and containing electrolytes and nutrients to be administered transdermally. The reservoir comprises multiple layers. The layer closest to the skin is microporous, thereby permitting substances to migrate from the reservoir to and from the skin. The microporous layer is followed by a pair of layers. The first layer of the pair consists of a water-soluble material, followed by a layer containing electrolytic and nutrient substances to be released. The water-soluble layer dissolves from contact with perspiration, thereby freeing the electrolytic and nutrient substances, allowing them to migrate through the holes to the skin. More pairs of layers may be situated behind such that small amounts of electrolytes are released to the skin over time in a controlled manner.

### BACKGROUND OF THE INVENTION

When a person exercises or performs athletic activities, he or she experiences greater than normal perspiration. For these activities, perspiration is necessary to promote heat loss. The rate of perspiration varies directly in proportion to the work rate as well as ambient temperature and humidity. It is well known that evaporation from a surface cools that surface. This is beneficial. However, during prolonged exercise, a person may become dehydrated. Perspiration mostly comprises water, but it also comprises necessary electrolytes ii and other nutrients essential for health and stamina, which are lost when a person perspires. Rehydration is necessary if the duration of exercise is greater than thirty minutes. While fluid intake of will satisfy this requirement for rehydration, inclusion of the electrolytes and nutrients in a liquid, such as a sports drink, is an inefficient method for allowing these substances to reenter the bloodstream. Optimally, other than replacement of lost water by drinking, the electrolytes can most effectively enter the bloodstream in the same way that they were lost—i.e., via absorption through the skin.

In 1971 and 1976, U.S. Pat. Nos. 3,589,122 and 3,948,254 were issued to Zaffaroni, respectively, for a bandage for administering drugs. This bandage is applied to the skin using an adhesive. Zaffaroni's bandage allows release of pharmaceutical substances to the skin through a permeable membrane that is in contact with the skin. In 1986, U.S. Pat. No. 4,597,961 was issued to Etscorn for a nicotine patch. Etscorn used a microporous membrane through which liquid nicotine would leech onto the skin. U.S. Pat. No. 8,182,908 was issued to Mrozinski in 2012 for a microporous membrane having a relatively large average pore size. In 2008, US Patent Application Publication Serial No. 2008/02863487 A1 by Todhunter disclosed a transdermal delivery system where a reservoir of medication comprises a medium separated from a user's skin by a dissolving layer. In 2010, US Patent Application Publication Serial No. 2010/0150996 A1 by Blitzer discloses a patch similar to Zaffaroni that could be used by athletes to replace electrolytes and essential nutrients during exercise.

All prior art references to adhesive patches that administer substances transdermally claim that these substances are administered over time. However, the timed release is governed only by the rate of absorption of these substances into an individual user's skin rather than by a controlled timed release. This is analogous to ingestion of Vitamin C (ascorbic acid) pills versus Timed Release Vitamin C capsules. When a person ingests a Vitamin C pill, the rate of absorption of Vitamin C into the bloodstream is determined by the amount of time for the pill to dissolve in the digestive tract plus the amount of time for the Vitamin ii C solution to migrate from the digestive tract into the bloodstream. Timed Release Vitamin C capsules comprise small globules of Vitamin C that dissolve at different rates. In this way, the distribution of Vitamin C takes place over a longer period. It would be desirable for those who exercise to be able to apply an adhesive patch that would release electrolytes and other nutrients into the bloodstream transdermally over a longer period in a controlled manner.

### SUMMARY OF THE INVENTION

The Present Invention is an adhesive patch applied to a person's skin much like a bandage. It comprises an impermeable material, the underside of which comprises an adhesive that permits the patch to adhere to the skin. Affixed to the adhesive underside is a reservoir containing the substances to be administered. The reservoir is initially covered with a peel-off synthetic material that prevents moisture from entering the reservoir. Prior to application to the skin, the peel-off synthetic material is removed. The patch is applied such that the reservoir is directly in contact with the skin.

The reservoir comprises multiple layers. The layer closest to the skin is microporous. This layer maintains the integrity of the reservoir and is insoluble. However, the microscopic holes permit substances to migrate from the reservoir to the skin. The rate of migration depends upon the microhole diameters. The next layer consists of a material that is water-soluble. The following layer contains the electrolytic and nutrient substances to be released. Water from perspiration enters the reservoir through the holes and causes the next layer to dissolve. This action frees the electrolytic and nutrient substances, thereby allowing them to migrate through the holes to the skin. Behind the electrolytic and nutrient substance layer are two more layers. The first of the two is another water-soluble material, and the second is another layer of electrolytes and nutrients. More pairs of layers may be situated behind such that small amounts of electrolytes are released to the skin over time in a controlled manner.

### BRIEF DESCRIPTION OF THE DRAWINGS

FIG. 1 illustrates a person running with an electrolyte-impregnated patch on his arm.

FIG. 2 is an illustration of a circular embodiment of the electrolyte-impregnated patch.

FIG. 2A is a top plan view of the circular embodiment.

FIG. 2B is a bottom plan view of the circular embodiment.

FIG. 3 is an illustration of a more-or-less rectangular embodiment of the electrolyte-impregnated patch.

FIG. 3A is a top plan view of the more-or-less rectangular embodiment.

FIG. 3A is a bottom plan view of the more-or-less rectangular embodiment.

FIG. 4 is a cross-sectional view of a portion of the electrolyte-impregnated patch.

### DETAILED DESCRIPTION OF THE INVENTION

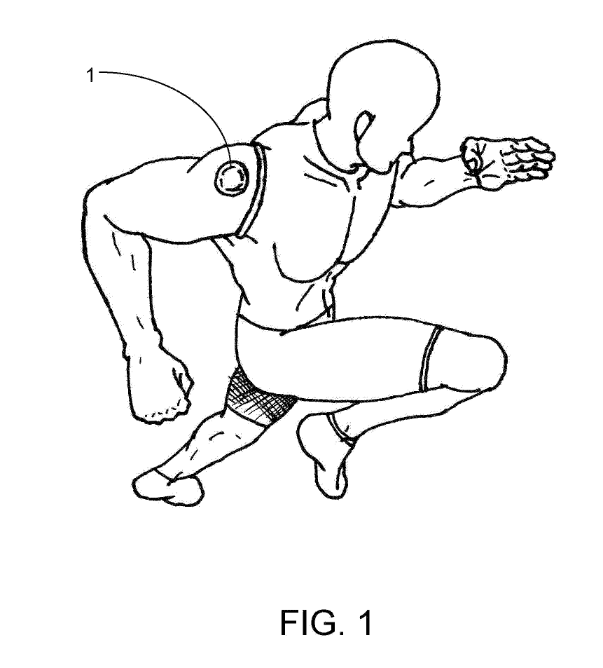

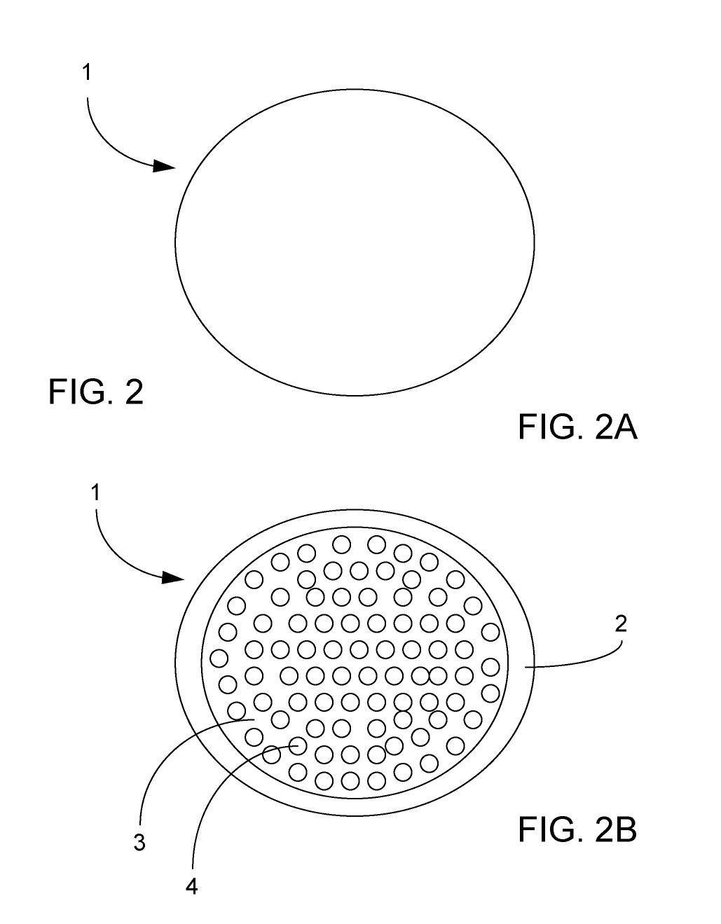

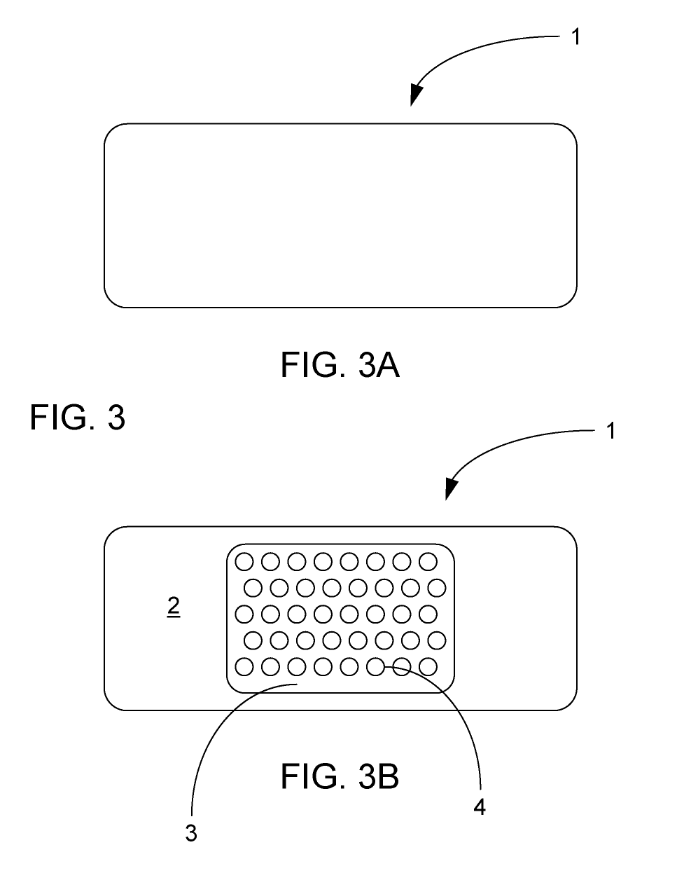

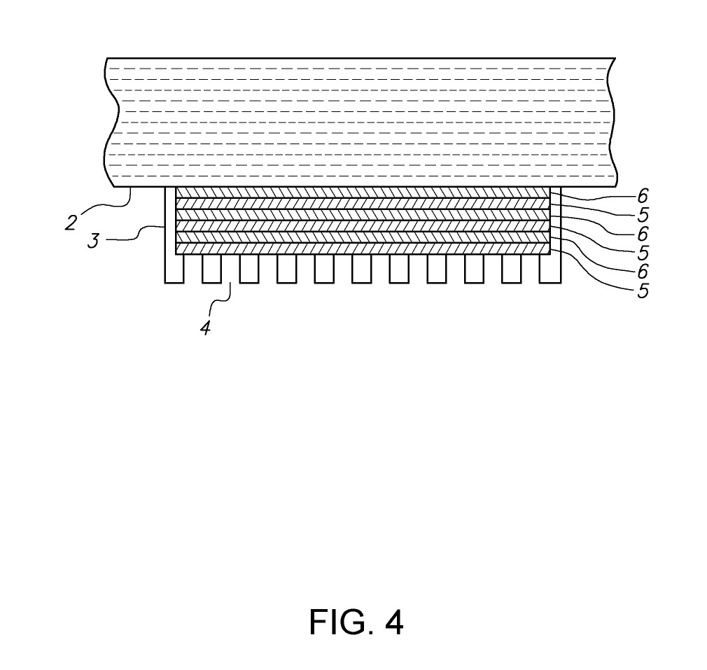

FIG. 1 shows an example of how a person performing exercises would apply the patch of the Present Invention. In the drawing, the running athlete applies patch, 1, to his arm. FIG. 2 shows patch 1 of the Present Invention in a circular embodiment, while FIG. 3 shows patch 1 in a more-or-less rectangular embodiment. The more-or-less rectangular embodiment resembles a Band-Aid®. The circle and rectangle are merely examples of the shapes of the patch. Other examples could include star shapes, sunburst shapes, team logos, etc. When purchased, the patch, 1, is preferably wrapped in a paper material that is impermeable to water. Removing the wrapper reveals the patch, 1, having a substrate comprising top and bottom surfaces. The substrate is insoluble in and impermeable to water. Adhering to the entire bottom surface is a water impermeable material that must be peeled off prior to use. Once this material is peeled off, the bottom surface becomes visible. Referring to FIG. 2B and FIG. 3B, this bottom surface comprises an adhesive 2 and a reservoir. The reservoir comprises a microporous outer layer 3. The bottom surface of the patch allows the outer layer 3 of the reservoir to be in contact with the user's skin once applied. The outer layer 3 comprises microscopic holes 4, through which perspiration can migrate from the skin into the reservoir and through which desired substances can migrate from the reservoir to the skin. FIG. 4 is a cross section of either the circular or the rectangular embodiment of patch 1. Within ii the reservoir are pairs of alternating layers of a water-soluble material 5 and a non-aqueous solution, suspension, or mixture of electrolyte and nutrient substances 6. There may be as many pairs of alternating layers 5 and 6 as desired. FIG. 4 shows three pairs of alternating layers. However, this is merely a convenient illustration.

Once unwrapped, the water impermeable material is peeled off the bottom surface, and the bottom surface of patch 1 is adhered to the user's skin with adhesive 2. As the person perspires, water from sweat permeates through the holes 4 in the reservoir's outer layer 3 to the water-soluble layer 5. Water-soluble layer 5 dissolves, thereby permitting the electrolytes and nutrients in layer 6 to migrate through the holes 4 to the user's skin, where they are absorbed into the bloodstream. This exposes the next water-soluble layer 5, which then dissolves into the sweat, thereby exposing the electrolytes and nutrients in the next layer 6, and so on. The number of layer pairs 5 and 6 would be designed such that the electrolytes could be released into the bloodstream over any desired time (e.g., uniformly over a four-hour period).

The electrolytes in layers 6 may comprise chlorides of sodium, potassium, and/or magnesium. The nutrients may comprise vitamins (e.g., Vitamins C or D) and/or amino acids (e.g., Taurine). Additional substances may comprise carbohydrates, chromium, anhydrous magnesium sulfate, dodecahydrated sodium hydrogen phosphate, dihydrogen phosphate, pyridoxine hydrogen chloride, dextrose, sucrose, sodium citrate, and/or citric acid. An energy enhancer, such as caffeine, could be included.

Materials that may be used to produce the primary substrate of the patch 1 and the associated adhesive 2 are well known in the art of bandage manufacture. The water-soluble layers 5 may comprise the following ingredients:
Pullulan—an edible polymer that is soluble in water;
Propylene Glycol—an emulsifier
Chondrus Crispus—a thickening agent
polysorbate 80—an emulsifier
ceratonia siliqua gum—a thickening agent

3M, ENTEK and Polystar Technologies, Inc. manufacture microporous polyethylene membranes that would be suitable for the reservoir's outer layer 3. The technology is described in U.S. Pat. No. 8,182,908.

Many state-of-the-art materials as well as newly developed materials may be used to manufacture the patch and its components. The electrolytes and nutrients in the reservoir disclosed above are only examples of the substances that can be administered transdermally into the bloodstream. Referring to FIG. 4, the novel aspect of the Present Invention is a reservoir encased in a microporous membrane, adhering to the active surface of a patch; said reservoir comprising alternate groups of layers each consisting of a water-soluble membrane and a non-aqueous mixture or suspension of the substances to be administered. Except for the non-aqueous mixture or suspension of said substances immediately adjacent to the top of the reservoir, each non-aqueous mixture or suspension is sandwiched between two water-soluble membranes. This structure enables the substances to be administered transdermally over a desired time period.

I claim:

1. A stick-on patch that permits substances to be transmitted into a person's bloodstream transdermally, said patch comprising:
a) a water-impermeable and insoluble substrate comprising a top surface and a bottom surface;
b) an adhesive affixed to the bottom surface of the substrate, wherein said adhesive enables the patch to adhere to the person's skin when applied; and
c) a reservoir comprising a top surface and a bottom surface, wherein said top surface of the reservoir is affixed to the bottom surface of the substrate, and wherein said reservoir further comprises:
i) a microporous membrane that encloses the reservoir, wherein said microporous membrane comprises a top surface and a bottom surface and further comprises microscopic holes through which liquid may pass;
ii) at least one pair of layers, wherein each pair comprises a water-soluble layer and a non-aqueous layer of the substances to be transmitted, wherein said water-soluble layer is a barrier preventing the substances from being transmitted;
whereby, when the patch is applied to the person's skin so as to adhere thereto, and the bottom surface of the microporous membrane maintains contact with the person's skin, and when the person perspires, perspiration will pass through the microscopic holes from the skin to the water-soluble layer, causing the water-soluble layer to dissolve in the perspiration, and the substances to be transmitted will be released, will pass through the microscopic holes to the skin, and will be absorbed transdermally into the person's bloodstream.

2. The patch of claim 1, wherein the at least one pair of layers in the reservoir comprises multiple pairs of layers.

3. The patch of claim 1, wherein the substances to be transmitted comprise electrolytes.

4. The patch of claim 1, wherein the substances to be transmitted comprise vitamins.

5. The patch of claim 1, wherein the substances to be transmitted comprise amino acids.

6. The patch of claim 1, wherein the substances to be transmitted are taken from the group consisting of sodium chloride, potassium chloride, magnesium chloride, Vitamin C, Vitamin D, L-Taurine, D-Taurine, carbohydrates, chromium, anhydrous magnesium sulfate, dodecahydrated sodium hydrogen phosphate, dihydrogen phosphate, pyridoxine hydrogen chloride, dextrose, sucrose, sodium citrate, and citric acid.

7. The patch of claim 1, wherein the substances to be transmitted comprise an energy enhancer.

8. The patch of claim 7, wherein the energy enhancer comprises caffeine.
